(19) United States
(12) Patent Application Publication
(10) Pub. No.: US 2008/0086176 A1
(43) Pub. Date: Apr. 10, 2008

(54) Method and Apparatus for Control of Pacemakers

(75) Inventor: Eran Ofek, Modi'in (IL)

Correspondence Address: EDWARD S. SHERMAN, ESQ., 3554 ROUND BARN BLVD., SUITE 303, SANTA ROSA, CA, 95403, US

(73) Assignee: Physical Logic AG, Zug (CH)

(21) Appl. No.: 11/868,518

(22) Filed: Oct. 7, 2007

Related U.S. Application Data
(60) Provisional application No. 60/828,754, filed on Oct. 9, 2006.

(51) Int. Cl. A61N 1/00

(52) U.S. Cl. 607/17

(57) ABSTRACT

A cardiac pacemaker device is in signal communication with a patient interface unit (PIU). The patient has the option of controlling various functions of the pacemaker via the PIU. In one embodiment, the pacemaker has an automatic means to detect a need for a change in pace rate, as well as initiate the calculated change. However, before or when initially increasing the pace rate the pacemaker device transmits an alert signal to the PIU. As the PIU alerts the patient that it believes they are engaging in exercise requiring a faster pacing rate, the patient can recognize a false signal and has the opportunity to transmit an override signal back to the pacemaker device (via the PIU), canceling the intended increase in pace. In other embodiments, the patient can use the PIU to notify the pacemaker that they are about to exercise, and optionally allow the pacemaker to work in automated mode or override the automated mode within reasonable limits to set a pacing rate commensurate with their physical activity.

### CROSS REFERENCE TO RELATED APPLICATIONS

The present application claims priority to the U.S. provisional application for a “Method and Apparatus for Control of Pacemakers” having Ser. No. 60/828,754 filed on Oct. 9, 2006, which is incorporated herein by reference

### BACKGROUND OF INVENTION

The invention generally relates to cardiac stimulation devices, and in particular to implanted cardiac pacemaker devices that have a means for varying the pacing rate based on the physiological demand for a minimal cardiac throughput of saturated blood.

By pacemaker we mean an artificial pacemaker, so as not to be confused with the heart's natural pacemaker (the sinus). Such pacemakers are medical devices that regulate the beating of the heart by providing electrical stimulation that replaces or overrides that of the hearts natural pacemaker, which if weakened or damaged would not supply the necessary electrical impulses to the heart. Alternatively, the native pacemaker may function properly, but the heart's normal electrical conduction pathways can be damaged to the extent that they impede the normal conduction of electrical impulses from the native pacemaker to the chambers of the heart, known as the atrium or ventricles.

Pacemakers, for the purpose of discussing the present invention, may be divided into two general groups: constant pace and rate responsive pacemakers. Pacemakers may also be differentiated by having a single lead or multiple leads, to pace just one chamber of the heart, and/or differentially pace different chambers on the heart, depending on the patient's condition and underlying pathology.

The constant pace devices are set at the implantation time to a specific pace which remains constant. In case the patient would like a different heart rate a new procedure is required. The main disadvantage for this kind of pacemaker is the fact that in case the patient is exerting himself physically then the pacemaker will not accelerate the heart beat accordingly, which can cause the patient to feel dizzy and even pass out.

The rate responsive pacemakers were developed to address this very significant disadvantage. This kind of device usually utilizes some sort of sensor to measure the physical activity of the patient (usually an accelerometer), the more acceleration (or movements) the sensor measures, the higher the heart beat the pacemaker sets and vice versa—no activity, keep a steady pace within normal parameters.

However, even the rate responsive pacemakers have a big disadvantage. The sensors they utilize don't necessarily detect the correct level of activity—for example, if a person is sitting in a car which bounces around a lot the pacemaker may mistake this for an elevated activity level and accelerate the heart rate without need—which may cause the patient physical symptoms such as dizziness etc. an additional example can be a patient riding his bicycle on a straight road, the fact that his upper body does not move a lot will cause the sensors to mistakenly think that the patient is not active and therefore will not be able to accelerate the heart rate as needed—which may cause the patient physical symptoms such as dizziness and loss of consciences.

It is therefore a first object of the present invention to provide a means for controlling a pacemaker after implantation.

It is therefore another object of present invention to provide a means to control or limit a rate responsive pacemaker by the patient after implantation.

### SUMMARY OF INVENTION

In the present invention, the first object is achieved by first providing in electrical communication with a patient a pacemaker device having a variable pace rate control, the pacemaker being capable of at least receiving an external signal that sets or modifies the pace rate. The patient is also provided with a patient interface unit (PIU) capable of transmitting an external signal to the pacemaker to control the pace rate. The patient is then able to select the pace rate, and then, using the PIU transmit the selected pace rate. The pacemaker receives this transmitted signal and changes the pace rate to that represented by the received signal.

Other objects of the invention are also achieved by providing with respect to a rate responsive pacemaker, which is otherwise of the kind described above, means to override or limit the automatic determination of the pacing rate from various sensors, and/or enable the automatic operation mode from the PIU.

In preferred embodiments, the PIU will send a command wirelessly to the pacemaker to either set a specific rate or set the minimum/maximum parameters for the pace the patient desires.

In other embodiments, the pacemaker will transmit an alert or warning to the PIU that it will change the pace rate unless overridden, and delay (with such delay time optionally being programmable) the change in pace rate for a predetermined time until the patient uses the PIU to cancel or modify the pending pace rate according to their actual level of activity or state of health.

Using acceptable medical practices the values of the pace or the minimum and maximum settings can be limited by the pacemaker and/or the PIU itself. For example, either device may prevent the patient from setting a value lower than the normal heart rate or higher that the recommended heart rate for physical activity.

As such the inventive device and method avoids unnecessary increases in heart rate when the patient is not exercising. It further assures adequate pacing when the patient is exercising. In other aspect of the invention, the PIU alerts the patients when the automated mode is not working properly. The device thus allows the patient to optimize the pacing rate more commensurate with their actual level of physical exertion.

The above and other objects, effects, features, and advantages of the present invention will become more apparent from the following description of the embodiments thereof taken in conjunction with the accompanying drawings.

### BRIEF DESCRIPTION OF DRAWINGS

FIG. 1A is a diagram of a patient equipped with an embodiment of the invention.

FIG. 1B is a schematic block diagram showing the components of a preferred embodiment of the device.

FIG. 2 is a process flow chart illustrating a method of using the device of FIGS. 1A and 1B.

FIG. 3 is a schematic block diagram showing the components of an alternative embodiment of the invention.

FIG. 4 is a process flow chart illustrating a method of using the device of FIG. 3.

### DETAILED DESCRIPTION

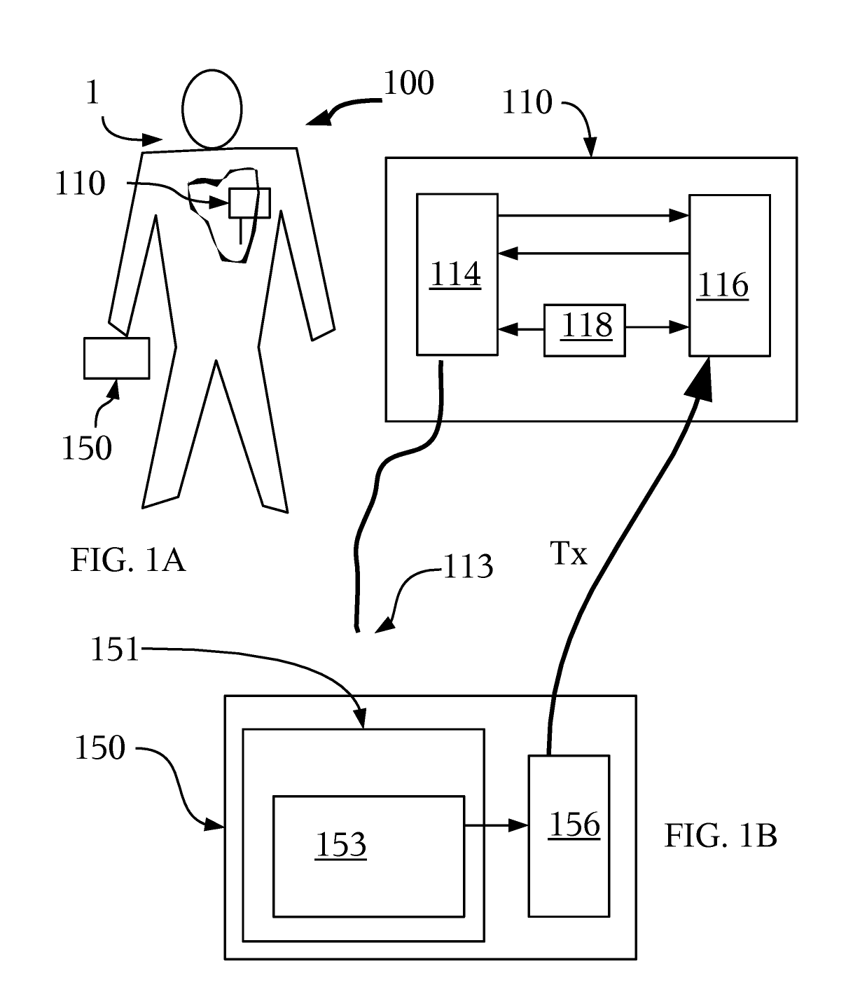

Referring to FIGS. 1 through 4, wherein like reference numerals refer to like components in the various views, there is illustrated therein a new and improved method and apparatus for control of rate responsive pacemakers, generally denominated 100 herein.

In accordance with the present invention, FIG. 1A is a diagram of a patient equipped with an embodiment of the invention. Device 100 includes a pacemaker 110 implanted in or attached to the patient 1. The patient 1 holds, is otherwise attached to or wears a Patient Interface Unite (PIU) 150. The pacemaker 110 and PIU 150 are in signal communication as will be further described. By pacemaker 110 we mean an artificial pacemaker, so as not to be confused with the heart's natural pacemaker. Such pacemakers are medical devices that regulate the beating of the heart by providing electrical stimulation that replaces that of the hearts natural pacemaker, which if weakened or damaged would not supply the necessary electrical impulses to the heart. Alternatively, the native pacemaker may function properly, but the heart's normal electrical conduction pathways can be damaged to the extent that they impede the normal conduction of electrical impulses from the native pacemaker to the chambers of the heart, known as the ventricles.

FIG. 1B is a schematic block diagram showing the components of an embodiment of the device 100 that includes the pacemaker 110 and PIU 150. The pacemaker 110 is either implanted or attached to the patient 1 and includes an internal pacemaker control 114 that is powered by a power source, such as a battery 118, generator or fuel cell, and the like. The pacemaker control 114 determines the power and frequency of electrical pulses delivered to the heart by the electrodes 113. The electrodes 113 may include one or more pairs of leads that have electrical terminals implanted or otherwise placed in specific locations to stimulate different regions of the heart, such as for example bi-ventricular pacing and the like.

The periodic or pulsed discharges from the terminals of electrodes 113 thus electrically stimulate at least specific portions of the cardiac muscle necessary for the targeted chamber(s) of the heart to cooperatively contract and hence pump blood. The specific placement of leads and electrical terminals, as well as the baseline setting of the timing, duration and power of discharges will be set by the physician according to the patient's diagnosis and conditions. Thus, the pacemaker control 114 is generally set at a nominal or baseline conditions for the aforementioned parameters prior to, or at the time of implantation.

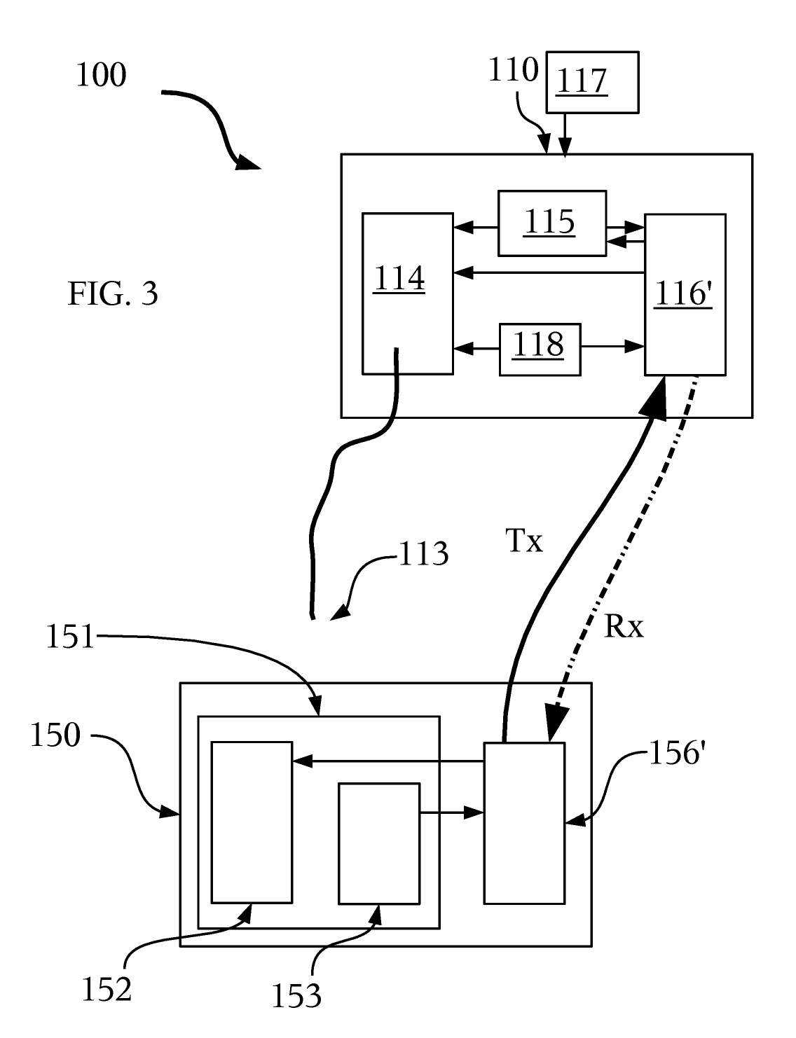

The pacemaker 110 includes at least a receiver 116 (and more preferably a transceiver 116′, as shown in FIG. 3) intended to communicate with the PIU 150. The PIU 150 also includes a transmitter 156, but more preferably in the case of the embodiment shown in FIG. 3, a transceiver 156′. The PIU 150 also includes a human interface device 151 that comprises at least an input device 153.

Thus, after the patient has recovered from the implantation surgery (if the pacemaker 110 is internal) when the patient has started to exercise or plans to start to exercise they use the input device 153 to regulate the pacing rate to the most appropriate level. In the case of a non-rate adaptive pacemaker the patient can set a specific pacing rate. In the case of a pacemaker with rate adaptive capabilities, the PIU can be used in multiple ways. These methods include, without limitation, overriding the automatic rate determination function to pace at the nominal fixed rate, or at least a higher, and possibly lower rate.

Alternatively, a rate responsive pacemaker 110 can be normally kept in a constant rate mode, until the patient plans to exercise, at which time they can use the PIU to place the pacemaker in the automatic mode. This method of use avoids the possibility of false readings during most of the day when they are not exercising. Such false readings can occur for example, if a person is sitting in a car which bounces around a lot, as the pacemaker may mistake this movement for an elevated activity level and accelerate the heart rate without need—which may cause the patient physical symptoms such as dizziness etc.

It should be understood that it is intended that the function of the device be limited to acceptable medical practices such that pace rate can only be set with a range having a minimum and maximum value. Such range can be limited by either the pacemaker 110 or the PIU 150. For example, the patient will be prevented from setting a value lower than the normal heart rate or higher that the recommended heart rate for physical activity.

Alternatively, even when the rate responsive pacemaker is in the automatic mode, the PIU can be used to place at least one of upper and lower bounds on the pacing rate that can be changed automatically. For example, if a patient is going to exercise by only moderate bicycle riding, but is concerned that a rough road might give a false positive reading (through excess vibration from the road surface) that they are exercising at a rate that requires a higher demand for oxygen, it would be useful to set an upper limit to the pace rate that is commensurate with intended moderate exercise level.

In any of the above methods of use, the PUI 150 will send a command (preferably wirelessly with an implanted pacemaker) via transmitter 156 to the receiver 116 of the pacemaker 110 to either set a specific rate, enable automatic rate determined pacing and/or set the minimum and maximum parameters for the desired pace rate as may be commensurate with the capabilities of the internal pacemaker control 114 of pacemaker 110.

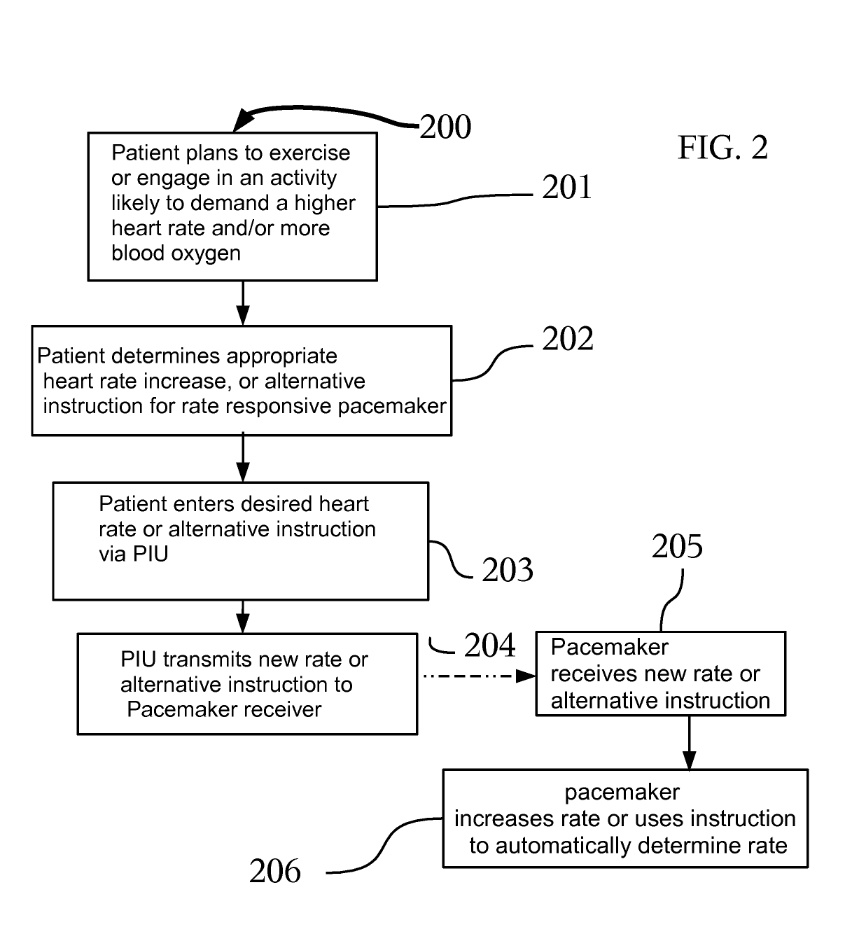

FIG. 2 is a process flow chart 200 showing how a patient would use device 100. Starting at step 201 the patient plans to exercise. Next in step 202, the patient determines the appropriate heart rate increase, or alternative instruction for a rate responsive pacemaker.

In step 203, the patient enters the desired heart rate or alternative instruction via PIU 150. The actuation of the patient's determination at this step may be as simply as turning a switch to a graphic indication of the type of exercise they plan to engage in, as the rate or rate limitation may be embodied in a pre-set condition represented by the same switch position.

In step 204, the PIU transmit new rate or an alternative instruction(s) to the pacemaker 110 via transmitter 156. Next, in step 205, pacemaker 110 receives the new rate or the alternative instruction(s) at receiver 116.

Then, in step 206 pacemaker control 114 in response to the information received at receiver 116 increases, decreases or uses instruction to automatically determine rate, as may be subject to a limitation embedded in the pacemaker 110 or sent from PIU 150.

In an alternative embodiment shown in FIG. 3, the pacemaker 110 has a transceiver 116′. As FIG. 3 is intended to represent a rate responsive pacemaker 110, the device 110 also includes at least one sensor 117 in signal communication with a computational unit 115 for the automated diagnostic analysis, using the output of the at least one sensor 117 to determine if the patient is undergoing non-sedentary activity. The computational unit 115 is generally also powered by battery 118 and is also in signal communication with both the transceiver 116′ and the pacemaker control 114. It should be appreciated that the function of the computational unit 115 and pacemaker control 114 may be embodied on a single general purpose computing device or dedicated microprocessor.

The computational unit 115, in addition to acquiring the output of sensor 117, also automatically determines the appropriate heart rate. Further, the computational unit 115 may also acquire additional information about the heart rate and the heart electrical activity useful in determining the appropriate power and location of pacing pulses or signal to be applied. Such computational unit may also include operating as a cardioverter, providing defibrillating or other therapeutic shocks to the patient on diagnosis of fibrillation or tachycardia. Thus, the computational unit 115 can automatically determine if the pacing rate should be adjusted upward or downward. The patient interface unit (PIU) 150 also includes a transceiver 156′. The PIU 150 further includes a human interface device 151 that comprises an alerting unit 152 and an input device 153. The alerting unit 152 is preferably non-visual stimulation, such as an audible, vibratory or other tactile alarm, but may also include a visual display interface to alert the patient that the computation unit 114 has detected a condition that under normally recognized medical practices would warrant the a change in the pace rate. While the PIU is preferably external, in other embodiments of the invention the PIU can be an implanted device to provide vibrational or electrical neural stimulation to alert the patient.

In one mode of operation, the pacemaker 110 transmits a warning to the PIU 150 and waits a predetermined time to before changing the pace rate. The PIU receives the warning and communicates it to the patient via the alerting unit 110. If the patient is conscious and has good reason to believe there is no need to increase the pace rate, i.e. the signal for increased/decreased oxygen demand is false, then they have the option of preventing the change in pace rate using the input device 153. The input device is preferably a tactile switch, which when activated by the patient, transmits via transceiver 156′ a signal to the pacemaker 110 directing the override of the pending increase in pace rate. Thus, if the pacemaker 110 receives the override signal within the predetermined time period from sending the warning to the PIU 150, the change in pace rate is cancelled. However, if say the patient is rendered drowsy or unconscious by the cardiac condition recognized by the computation unit as requiring more oxygenated blood, or is otherwise distracted by their actual exercise or exertion, they will either be unable to activate the input device or simply ignore it and will then have their pace rate increased or decreased according to the direction of the computational unit 115.

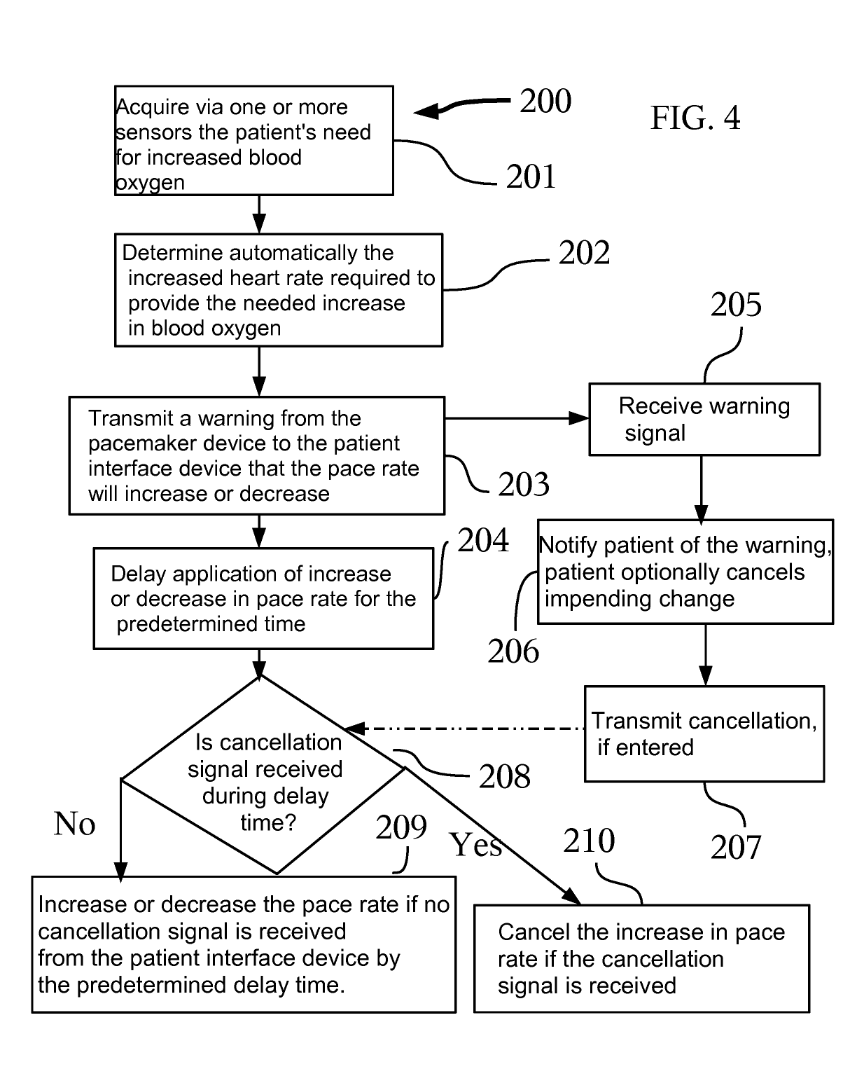

FIG. 4 is a process flow chart 200 further defining one method of using device 100 in FIG. 3. In the first step 201 the pacemaker 110, via computational unit 115, acquires an indication of the patient's potential need for more blood oxygen via one or more sensors 117.

In the next step 202, depending on the patient's physical condition the computation unit 115 determine automatically that the pace rate of the patient 1 should be increased or decreased to a particular value from the current rate.

Then, in step 203 the pacemaker 110 transmits a warning to the PIU 150 that a change in pace rate is imminent, that is it will be applied within a predetermined period of time if not cancelled by the patient. Step 204 is shown as delaying the change in pace rate by the pacemaker 110 by the predetermined time while awaiting to receive a cancellation signal from the PIU 150 (step 207)

In the next step 205, the warning signal, if any, is received at the PIU transceiver 156′. Then in step 206 the patient 1 if conscious, will be alerted via the alerting unit 152.

If conscious, the patient 1 may then determine that the change in pace is not necessary and does not want their heart rate changed. Thus, using via interface unit 151 the patient 1 will use the input device 153 to cancel the impending change in pace rate. Thereafter in step 207, the PIU then generates and transmits the cancellation signal to the pacemaker device 110.

Then, in step 208 the computational unit 115 determines if the cancellation signal has been detected by the transceiver 116′ during delay time. The alternative outcomes leading to step 209 or 210 respectively are the change in pace rate (step 209) if no cancellation signal is received, and the cancellation of the change in pace rate (step 210) if the cancellation signal is received in step 208.

Other embodiments of the invention include the ability of the patient or clinician to set the predetermined time (in step 204) for future pending changes in pace rate via the PIU 150.

In other embodiments of the invention, it is preferable that at least one of the pacemaker 110 and the PIU 150 can be programmed to limit the override functions under predetermined conditions. For example, under conditions of apparently extreme need for increased blood oxygen that could not possibly be explained as errors or artifacts it would be desirable to immediately change the pace rate of the patient and avoid any delay, whereas for more uncertain or borderline diagnosis it would be preferably to provide the patient the opportunity to override or cancel the change in pace rate as envisioned in steps 203-210 of FIG. 2.

In another embodiment of the invention potential modes of using or initializing the PIU 150, such as the delay time or the conditions when the override would not be possible, can only be set by the patient's physician, and not the patient. A more preferred embodiment of the invention may include providing a password or related secured code means for modifying the operational parameters of at least one of the PIU 150 and pacemaker 110 or device 100. In another aspect of the invention, the automatic or alert modality of pacemaker 110, described with respect to FIG. 4 is varied via PIU 150.

Another aspect of the invention includes providing means to store, which is a data log, the conditions under which changes in pace rate were predicted and applied or cancelled by the patient, including output means for clinician use. Such data logging provides a history of the conditions under which changes pace rate were determined as appropriate by the computational unit 115 and applied or cancelled by the patient 1. Having such a history is expected to allow the physician to more appropriately adjust the automatic diagnostic parameters of the pacemaker 110, such that a lower number of false positive indications of a need for a change in pace rate would occur and need to be canceled or overridden by the patient.

Another preferred aspect of the invention includes the diagnostic function of the pacemaker 150 being automatically responsive to the logged history of the when changes in the pace rate were determined as appropriate by the computational unit 115 and either allowed to be applied or cancelled by the patient 1. That is, the logic control of computation unit 115 “learns” from the patient override history to better distinguish between false positive readings and actual abnormalities that require a change in pace rate.

It will of course be appreciated that when the pacemaker device 110 is internal or implanted in the patient the transmitter, receiver and/or transceiver communication means between the pacemaker 110 and the PIU 150 is preferably wireless, with protocols that might include the Bluetooth standard, and the like.

In another embodiment of the invention, at least one of the pacemaker 110 and the PIU 150 can be programmed with conditions that override or prevent the override functions of steps 203 to 208. Such predetermined conditions to limit the override functions may include frequency of overrides per unit of time (i.e. 1×/3 hours) as well as specific sensor and/or ECG parameters, and the like.

While the invention has been described in connection with a preferred embodiment, it is not intended to limit the scope of the invention to the particular form set forth, but on the contrary, it is intended to cover such alternatives, modifications, and equivalents as may be within the spirit and scope of the invention as defined by the appended claims.

1. A method for cardiac pacing, the method comprising the steps of:
a) providing in electrical communication with a patient a pacemaker device having a variable pace rate control, the pacemaker capable of at least receiving an external signal that sets or modifies the pace rate,
b) providing a patient interface unit (PIU) capable of transmitting an external signal to the pacemaker to control the pace rate.
c) selecting a pace rate,
d) transmitting the pace rate selected from the PIU,
e) receiving the transmitted signal at the pacemaker,
f) changing the pace rate to that represented by the received signal.

2. A method for cardiac pacing, the method comprising the steps of:
a) providing in electrical communication with a patient, a pacemaker device having a variable pace rate control that is responsive to an automated diagnostic analysis of at least one sensor to determine if the patient is undergoing non-sedentary activity,
b) providing a patient interface device capable of at least one of receiving information on the status of the automated diagnostic analysis of the pacemaker and transmitting control signals that modify the function of the pacemaker to either override or permit the control thereof by the automated diagnostic analysis
c) communicating at least one of the status of the automated diagnostic analysis of the at least one sensor of the pacemaker and control signals between the pacemaker and the patient interface device.

3. The method of claim 2 wherein said step of communicating is the duplex communication of control signals from the patient interface unit and the status of the automated diagnostic analysis of the pacemaker.

4. The method of claim 2 wherein the communication of control signals from the patient interface unit modulates the function of the automated diagnostic analysis from a constant rate to automated variable rate control in response to the sensor thereof.

5. The method of claim 2 wherein the communication of control signals from the patient interface unit modulates the function of the automated diagnostic analysis from automated variable rate control in response to the sensor thereof to constant rate.

6. The method of claim 2 further comprising the steps of:
a) determining automatically from the at least one sensor signal that a change in pace rate should be applied to the patient,
b) transmitting an alert from the pacemaker device to the patient interface device that a change in pace will or has occurred,
c) receiving the transmitted signal at the patient interface device pacemaker,
d) communicating the new actual or incipient change in pace rate to the patient.

7. A method according to claim 6 wherein the change of pace rate is delayed by a predetermined time while awaiting to receive a cancellation signal from the patient interface device and further comprising the step of:
a) changing the pace rate if no cancellation signal is received from the patient interface device by the predetermined time.

8. The method of claim 3 further comprising the step of data logging the past history of duplex communications between the patient control unit and the pacemaker.

9. The method of claim 7 further comprising the step of data logging the past history of automated diagnostic analysis that was cancelled by the patient.

10. The method of claim 9 wherein the data log of the history of the automated diagnostic analysis that was cancelled by the patient is automatically analyzed to reduce the incidence of providing additional alerts that are likely to be cancelled by the patient.

11. The method of claim 2 wherein the patient interface unit transmits control signals that limit at least one of the minimum and maximum pace rate that can be set by the variable pace rate control.

12. The methods of claim 5 wherein the patient has modulated the automated rate control to a constant rate and further comprising the steps:
a) detecting via the sensor a condition that requires the pacemaker to revert back to setting a pace rate by the automated diagnostic analysis,
b) modulating the pace rate in response to the automated diagnostic analysis.

13. The method of claim 7 wherein the patient interface unit is used to program the pacemaker with conditions that override the effect of transmission of a cancellation signal by the patient.

14. The method of claim 2 wherein a code must be entered into the patient interface unit before it can be used to transmit control signals to the pacemaker.

15. A system for controlled and rate responsive cardiac pacing, the system comprising:
a) a pacemaker device that includes,
i) at least one pacing lead in electrical communication with the heart muscle to apply a heart pacing pulse,
ii) at least one sensor to determine if the patient is undergoing non-sedentary activity,
iii) a first transceiver,
iv) a computation unit in signal communication with the at least one sensor to determine the patient's apparent need for a change in pace rate, and further in signal communication with the first transceiver whereby the first transceiver is capable of the transmitting information about the actual or an incipient change in pace rate and receiving at least an external signal,
v) at least one power source to power one or more of the pacing lead, sensor, transceiver and computation unit, said at least one power source output being responsive to the computation unit to deliver a periodic pacing discharge at the pacing lead unless interrupted by an external signal received from the transceiver,
b) a patient interface unit having;
i) a second transceiver in signal communication with the first transceiver,
ii) a human interface device in signal communication with the second transceiver to transmit to the patient the actual or incipient pace rate of the pacemaker,
iii) whereby the patient interface unit is responsive to the patient to cause the second transceiver to send an external signal to the first transceiver, and further whereby the external signal that modulates the function of the pacemaker.

16. A system for controlled and rate responsive cardiac pacing according to claim 15 wherein the external signal from the second transceiver causes the computation unit of said pacemaker to modulate the pace rate from that computed from the information received from the sensor

17. A system for controlled and rate responsive cardiac pacing according to claim 15 wherein the external signal form the second transceiver causes the computation unit of said pacemaker to revert to a constant pace rate mode.

18. A system for controlled and rate responsive cardiac pacing according to claim 15 wherein the external signal form the second transceiver causes the computation unit of said pacemaker to change from a constant pace rate mode to an automated mode wherein the pace rate is determined by the computation unit.
